(19) United States
(12) Patent Application Publication
(10) Pub. No.: US 2023/0364192 A1
(43) Pub. Date: Nov. 16, 2023

(54) MODULATION OF BETA CELL REGENERATION THROUGH GPR75 SIGNALING

(71) Applicant: New York Medical College, Valhalla, NY (US)

(72) Inventors: Ercument Dirice, Valhalla, NY (US); Danielle Diegisser, Valhalla, NY (US); Michal Schwartzman, Valhalla, NY (US)

(21) Appl. No.: 18/314,438

(22) Filed: May 9, 2023

Related U.S. Application Data
(60) Provisional application No. 63/340,106, filed on May 10, 2022.

(51) Int. Cl. A61K 38/19; C07K 16/28; A61K 31/202; A61P 3/10

(52) U.S. Cl. CPC A61K 38/195; C07K 16/28; A61K 31/202; A61P 3/10

(57) ABSTRACT

The present disclosure provides methods for regulating GPR75 responses in islet β-cells by administering to a mammal a compound that activates the activity of GPR75 expressed in β-cells of the pancreas. The methodology described herein can be used for increasing the number of β-cells in the pancreatic islets for use in treatment of metabolic disorders such Type-I or Type-II diabetes.

This application claims benefit and priority to U.S. Provisional Application No. 63/340,106 filed on May 10, 2022, which is incorporated herein by reference in its entirety.

### FIELD OF THE INVENTION

The present disclosure provides methods for stimulating the proliferation of pancreatic β-cells by administering to a mammal a compound that activates the GPR75 receptor. The methodology described herein can be used for increasing the number of β-cells in the pancreas for use in treatment of metabolic disorders such Type-I or Type-II diabetes.

### BACKGROUND OF THE INVENTION

Islet beta cells are a type of cell found in pancreatic islets that synthesize and secrete insulin and amylin. Beta cells make up 50-70% of the cells in human islets. In patients with Type 1 or Type-II diabetes, beta-cell mass and function are diminished, leading to insufficient insulin secretion, hyperglycemia and many secondary detrimental pathologies. Accordingly, novel methods are needed that can act to stimulate the proliferation of β-cells within the pancreas.

### SUMMARY OF THE INVENTION

The present disclosure provides methods for regulating GPR75 mediated responses in pancreatic β-cells by administering to a mammal a compound that activates GPR75 expressed within β-cells of the pancreas. The methodology described herein can be used for increasing the number of β-cells in the pancreas for use in treatment of metabolic disorders such Type-I or Type-II diabetes. According to the present disclosure, this activation may be achieved by any compound that activates proliferation of GPR75 expressing β-cells.

More specifically, the presently disclosed subject matter provides a method of treating, delaying the onset, alleviating a symptom, and/or preventing the progression of metabolic disorders such as Type-I or Type-II diabetes. The method includes administering safely to a subject in need thereof a therapeutically effective amount of a GPR75 agonist. The administration of such GPR75 agonists is intended to, as demonstrated herein, modulate the proliferation of ß-cells. Targeted cells are those ß-cells found within the pancreas. While the disclosure below is directed as a treatment of Type-I or Type-II diabetes, the disclosure applies equally as well to treating, delaying the onset, alleviating a symptom, and/or deterring the progression of other metabolic diseases that result from an insufficient number of ß-cells found within the pancreas.

The presently disclosed subject matter provides a method for preventing, treating or alleviating, the symptoms associated with Type-I or Type-II diabetes through administration of a GPR75 agonist in a therapeutically effective amount to a subject suspected of suffering from, or at risk of developing Type-I or Type-II diabetes. Such subjects may be identified as those having the symptoms of Type-I or Type-II diabetes. Symptoms of diabetes include, for example, excessive urination, thirst, hunger, loss of weight, blurred vision, numb or tingling hands or feet, lethargy, and dry skin.

In an embodiment, the subject to be treated may be one suffering from disorders or conditions that render a subject more prone for development of metabolic disorders such as Type-I or Type-II diabetes. Such disorders or conditions also include, but are not limited to, obesity.

Pharmaceutical compositions and formulations for use in treatment of Type-I or Type-II diabetes include pharmaceutical compositions of a GPR75 agonist, alone or in combination with one or more additional therapeutic agents, in a mixture with a physiologically compatible carrier, which can be administered to a subject, for example, a human subject, for therapeutic treatment. The presently disclosed pharmaceutical compositions can be administered using a variety of methods known in the art depending on the subject and/or the severity of the Type-I or Type-II diabetes. In an aspect, the GPR75 agonist is administered, for example, orally, intranasally, by inhalation or intravenously.

The presently disclosed subject matter also includes the use of a GPR75 agonist, in the manufacture of a medicament for treatment of Type-I or Type-II diabetes. Regardless of the route of administration selected, the GPR75 agonist pharmaceutical compositions are formulated into pharmaceutically acceptable dosage forms such as described below or by other conventional methods known to those of skill in the art.

Actual dosage levels of the GPR75 agonist can be varied to obtain an amount of the active ingredient that is effective to achieve the desired therapeutic response for a particular subject without being toxic to the subject. The selected dosage level will depend on a variety of factors including the route of administration, the time of administration, the rate of excretion, the duration of the treatment, other drugs used in combination with the GPR75 agonist, the age, sex, weight, condition, general health, and prior medical history of the patient being treated, and like factors well known in the medical arts. A physician having ordinary skill in the art can readily determine and prescribe the effective amount of a given GPR75 agonist-containing pharmaceutical composition.

The presently disclosed compositions of a GPR75 agonist can be assembled into kits or pharmaceutical systems for use in treating Type-I or Type-II diabetes. In some embodiments, the presently disclosed kits or pharmaceutical systems include a GPR75 agonist in unit dosage form. In further embodiments, the GPR75 agonist can be present together with a pharmaceutically acceptable solvent, carrier, excipient, or the like, as described herein. In some embodiments, the presently disclosed kits include one or more containers, including, but not limited to a vial, tube, ampule, bottle, and the like, containing the GPR75 agonist. The presently disclosed kits or pharmaceutical systems also can include associated instructions for using the GPR75 agonist containing compositions for the treatment of Type-I or Type-II diabetes.

### BRIEF DESCRIPTION OF THE DRAWINGS

FIG. 1A-C. Pancreatic islet size is reduced in GPR75KO mice: FIG. 1A. Representative H&E images of pancreatic sections from 8-week-old control and GPR75KO mice. FIG. 1B. Quantification of average islet area. FIG. 1C. Quantification of islet diameter. Islet diameter is calculated as the average of the longest and shortest diameter for each islet. All islets in each pancreas were analyzed. Arrows show pancreatic islets. Scale bar: 100 μm. **P<0.01 and ***P<0.001

FIG. 2A-B. Total islet area is reduced in GPR75KO mice: FIG. 2A Representative H&E images of pancreatic sections from 8-week-old control and GPR75KO mice. FIG. 2B Quantification of total islet area per pancreas. Arrows show pancreatic islets. Scale bar: 100 μm. **P<0.01.

FIG. 3A-B. Total numbers of islets are reduced in GPR75KO mice: FIG. 3A. Representative H&E images of pancreatic sections from 8-week-old control and GPR75KO mice. FIG. 3B. Quantification of total islet numbers per mm2 pancreas area. All islets in each pancreas were analyzed. Arrows show pancreatic islets. Scale bar: 100 μm. *P<0.05.

FIG. 4A-D. Adaptive β cell mass is impaired in GPR75KO mice in response to high-fat diet: FIG. 4A. Representative H&E images of pancreatic sections from 24-week-old HFD-fed (16-weeks) control and GPR75KO mice. Scale bar: 100 μm. FIG. 4B. Quantification of average islet area. FIG. 4C. Representative H&E images of pancreatic sections from 24-week-old HFD-fed (16-weeks) control and GPR75KO mice. Images show most abundant islet areas of each pancreas. Scale bar: 500 μm. FIG. 4D. Quantification of total islet area per pancreas. All islets in each pancreas were analyzed. Arrows show pancreatic islets. **P<0.01.

FIG. 5A-B. Total numbers of islets are reduced in HFD-fed GPR75KO mice: FIG. 5A. Representative H&E images of pancreatic sections from 24-week-old HFD-fed (16-weeks) control and GPR75KO mice. FIG. 5B. Quantification of islet numbers. All islets in each pancreas were counted. Scale bar: 100 μm. *P<0.05

FIG. 6A-B. β-cell proliferation is reduced in HFD-fed GPR75KO mice: FIG. 6A. Representative images of pancreatic sections obtained from 24-week-old HFD-fed (16-weeks) control and GPR75KO mice stained for insulin (red), proliferation marker Ki67 (green) and nuclear marker DAPI (blue). Insets on the left point to Ki67+β-cells. Insets on the right show individual staining for the proliferating β-cells depicted in the square. FIG. 6B. Quantification of Ki67+ β cells. **P<0.01

### DETAILED DESCRIPTION OF THE INVENTION

The present disclosure provides for a method for activating the proliferation of islet β-cells within a subject which comprises administering to said subject an activating amount of a GPR75 agonist (alternatively termed herein a GPR75 agonist or a GPR75 receptor agonist). This disclosure provides a method for increasing the number of β-cells, for stimulating β-cell proliferation and for preventing or ameliorating diabetes. Proliferation as used herein refers to an increase in cell number, for example by cell division. The invention is based on the recognition that GPR75 acts as an β-cell growth factor receptor. The invention also relates to a method for treating or preventing Type I or Type II diabetes, a method for obtaining a less severe disease stage in a subject suffering from Type I or Type II diabetes as well as methods of delaying the progression of impaired glucose tolerance (IGT) or non-insulin requiring Type I or Type II diabetes to insulin requiring Type I or Type II diabetes.

In an embodiment, the GPR75 agonist can be administered using a variety of methods known in the art. More particularly, the GPR75 receptor agonist can be administered by any suitable route of administration, including administration methods such as topical, parenteral, intramuscular, nasal, oral, transdermal, mucosal, and subcutaneous or other modes of delivery known in the art.

Provided methods are useful for the treatment and/or prevention of metabolic disorders such as Type I or Type II diabetes. As used herein, the terms “treat”, “treating”, “treatment” and the like, are meant to decrease, suppress, attenuate, diminish, arrest, the development or progression of the metabolic disorders and/or symptoms associated therewith. The terms “treat”, “treating”, “treatment” and the like, as used herein can refer to curative therapy, prophylactic therapy, and preventative therapy. Accordingly, as used herein, “treating” means either slowing, stopping or reversing the progression of the metabolic disorders and/or symptoms associated therewith.

As used herein, the terms “prevent”, “preventing”, “prevention”, “prophylactic treatment” and the like refer to reducing or inhibiting the probability of developing symptoms of the metabolic disorder in a subject. Thus, in some embodiments, a GPR75 receptor agonist can be administered prophylactically to prevent the onset of metabolic disorders or to prevent the recurrence of metabolic disorders in a subject.

The subject of the inventive methods may be a human or a non-human animal. Non-limiting examples of non-human animals that may be used in the methods according to the invention include companion animals, such as dogs or cats, and large animals such as those used in animal husbandry (for example, sheep, cattle, pigs, llamas, buffalo etc.) or wild animals such as tigers, lions, elephants, etc.

A number of methods, well known to those of skill in the art, may be used for identifying modulators of β-cell number (such as proliferation), for example, proteins that increase β-cell number and/or stimulate β-cell proliferation through activation of GPR75. In some embodiments, when using animal models the presence or amount of β-cell in the pancreas can be determined by histological staining, in situ hybridization, flow cytometry, or immunohistochemistry. In one particular example, the presence or amount of β-cells is determined by detection of one or more proteins expressed by mature β-cells (for example, specifically expressed by β-cells but not by other pancreatic cells), such as insulin, MafA, Pdx1, Nkx2.2, diacylglycerol kinase beta (DGKB), or glycoprotein M6A (GPM6A) (see, e.g., Dorrell et al., Mol. Cell. Endocrinol. 339:144-150, 2011). One of ordinary skill in the art can identify additional markers for detection of presence and/or amount of β-cells.

In some examples, the number of β-cells or the amount of proliferation of β-cells in a test animal contacted or treated with a test compound, i.e., a possible GPR75 agonist, is compared to a control. A “compound” or “test compound” is any substance or any combination of substances that is useful for achieving an end or result. Any compound that has the potential to modulate β-cell number and/or proliferation through activation of GPR75 can be tested using the methods of this disclosure.

Exemplary test compounds include, but are not limited to, peptides, such as soluble peptides, including but not limited to members of random peptide libraries, antibodies and antibody fragments and small organic or inorganic molecules. Appropriate test compounds can be contained in libraries, for example, synthetic or natural compounds in a combinatorial library. Numerous libraries are commercially available or can be readily produced; means for random and directed synthesis of a wide variety of organic compounds and biomolecules also are known. Alternatively, libraries of natural compounds in the form of bacterial, fungal, plant and animal extracts are available or can be readily produced. Additionally, natural or synthetically produced libraries and compounds are readily modified through conventional chemical, physical and biochemical means, and may be used to produce combinatorial libraries. Such libraries are useful for the screening of a large number of different compounds for identification of GPR75 agonists.

In screening methods, the number of β-cells is measured by counting the number of β-cells in the pancreas of a test subject or animal. Methods for counting β-cells include manual counting (for example examining a sample (such as tissue or an organism under a microscope) and counting the number of β-cells). β-cells can be identified by staining techniques, including histological stains (such as hematoxylin and eosin) and immunohistochemistry or in situ hybridization (for example, using β-cells-specific antibodies or probes (such as for insulin, diacylglycerol kinase beta, or glycoprotein M6A)). An increase in the number of β-cells and/or proliferation rate (such as an increase of at least about 0.5%, 1%, 2%, 5%, 10%, about 20%, about 50%, about 80%, about 90%, about 1.5-fold, about 2-fold, about 3-fold, about 5-fold, about 10-fold or more proliferation rate) in the presence of one or more test compounds as compared to in the absence of the one or more test compounds indicates that the compound increases β-cell number and/or proliferation.

In other examples, β-cell proliferation is measured using methods of measuring cell proliferation which are known to one of ordinary skill in the art. Such methods include in vitro or in vivo methods. In some examples, cell proliferation is measured by incorporation of a DNA label (for example 5-bromo-2-deoxyuridine (BrdU), 5-ethynyl-2′-deoxyuridine, (EdU) or ³[H]-thymidine). An increase in detection of the number of labeled cells (such as an increase of about 0.5%. 1%, 2%, 5%, 10%, about 20%, about 50%, about 80%, about 90%, about 1.5-fold, about 2-fold, about 3-fold, about 5-fold, about 10-fold or more) in the presence of one or more test compounds as compared to in the absence of the test compounds indicates that the compound increases β-cell proliferation.

In other examples, β-cell proliferation is measured by detecting cellular DNA content in a population of cells, as DNA content is closely proportional to cell number. Such methods include detecting a dye that binds to nucleic acids (such as CYQUANT cell proliferation kit, Invitrogen). In other examples, cell proliferation is measured by quantifying cleavage of a tetrazolium salt (such as MTT, XTT, or MTS) to insoluble formazan crystals by mitochondrial dehydrogenase.

Glucose levels may also correlate with the number and/or proliferation of β-cells. Therefore, in still further examples, the effect of a test compound on number and/or proliferation of β-cells can be determined by measuring glucose levels in a test subject or animal treated with the test compounds as compared in the absence of the test compounds.

In an embodiment, compositions are provided comprising a GPR75 receptor agonist that enhances the proliferation of β-cells. Such proliferation, as demonstrated below can result in an increase in islet, size, area and number. The administration of such compositions is intended to induce the activity of GPR75 in the subject to be treated thereby stimulating the proliferation of β-cells.

Non-limiting examples of the GPR75 receptor agonists that can be used in the present invention include, for example, C-C Motif Chemokine Ligand 5 (CCL5) and 20-hydroxyeicosatetraenoic acid (20-HETE).

As used herein, in general, a “therapeutically effective amount” of a GPR75 agonist refers to the amount of the agent necessary to elicit the desired biological response. In a specific embodiment, an effective amount is an amount sufficient for prevention or treatment of Type-I or Type-II diabetes and associated symptoms.

The effective amount of an agent may vary depending on such factors as the desired biological endpoint, the composition of the pharmaceutical composition, the target tissue Pancreatic islets or cell (β-cells), the health of the subject to be treated and the like. In some embodiments, the term “therapeutic effective amount” refers to an amount sufficient to reduce or ameliorate the severity, duration, progression, or onset of symptoms associated with Type-I or Type-II diabetes.

A “subject” can include a human subject for medical purposes, such as for the treatment of Type-I or Type-II diabetes or the prophylactic treatment for preventing the onset of Type-I or Type-II diabetes, or an animal subject for medical, veterinary purposes, or experimental purposes. Further, a “subject” can include a patient afflicted with or suspected of being afflicted with Type-I or Type-II diabetes. Thus, the terms “subject” and “patient” are used interchangeably herein. In one embodiment, the presently disclosed subject matter relates to a method of treating or preventing Type-I or Type-II diabetes in a subject in need thereof, the method including administration to the subject of a therapeutically effective amount of a GPR75 agonist.

As used herein, “Type-I or Type-II diabetes” means that the subject has symptoms associated with Type-I or Type-II diabetes. Such Type-I or Type-II diabetes symptoms include, for example, excessive urination, thirst, hunger, loss of weight, blurred vision, numb or tingling hands or feet, feeling tired and dry skin.

As used herein, the terms “treat,” treating,” “treatment,” and the like, are meant to decrease, suppress, attenuate, diminish, arrest, the underlying cause of the Type-I or Type-II diabetes or to stabilize the development or progression of the Type-I or Type-II diabetes and/or symptoms associated therewith. The terms “treat,” “treating,” “treatment,” and the like, as used herein can refer to curative therapy, prophylactic therapy, and preventative therapy. Accordingly, as used herein, “treating” means either slowing, stopping or reversing the progression of Type-I or Type-II diabetes, including reversing the progression to the point of eliminating the symptoms of Type-I or Type-II diabetes. It should be appreciated that treating a disease, disorder or condition does not require that the disease, disorder, condition, or symptoms associated therewith be completely eliminated.

As used herein, the terms “prevent,” “preventing,” “prevention,” “prophylactic treatment” and the like refer to reducing the probability of developing symptoms of Type-I or Type-II diabetes in a subject, who does not have, but is at risk of or susceptible to developing Type-I or Type-II diabetes. Thus, in some embodiments, a GPR75 agonist can be administered prophylactically to prevent the onset of Type-I or Type-II diabetes or to prevent the recurrence of Type-I or Type-II diabetes in a subject.

The treatment, administration, or therapy can be continuous or intermittent. Continuous treatment, administration, or therapy refers to treatment on at least a daily basis without interruption in treatment by one or more days. Intermittent treatment or administration, or treatment or administration in an intermittent fashion, refers to treatment that is not continuous, but rather cyclic in nature. Treatment according to the presently disclosed methods can result in complete relief or cure from Type-I or Type-II diabetes or partial amelioration of one or more symptoms of Type-I or Type-II diabetes and can be temporary or permanent.

In certain embodiments, the presently disclosed subject matter also includes combination therapies. Additional therapeutic agents, which are normally administered to treat or prevent Type-I or Type-II diabetes, may be administered in combination with a GPR75 agonist as disclosed herein. For example, the GPR75 agonist may optionally be administered in conjunction with other compounds (e.g., therapeutic agents) or treatments useful in treating Type-I or Type-II diabetes. These additional agents may be administered separately, as part of a multiple dosage regimen, from the composition comprising a GPR75 agonist as disclosed herein. Alternatively, these agents may be part of a single dosage form, mixed together with a GPR75 agonist, in a single composition.

By “in combination with” is meant the administration of a GPR75 agonist, with one or more therapeutic agents either simultaneously, sequentially, or a combination thereof. Therefore, a subject can be administered a combination of a GPR75 agonist and one or more therapeutic agents at the same time (i.e., simultaneously) or at different times (i.e., sequentially, in either order, on the same day or on different days), as long as the effect of the combination of both agents is achieved in the subject. Where the GPR75 agonist and one or more therapeutic agents are administered simultaneously, they can be administered to the subject as separate pharmaceutical compositions, each containing either a GPR75 agonist or one or more therapeutic agents or be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times. In such combination therapies, the therapeutic effect of the first administered agent is not diminished by the sequential, simultaneous or separate administration of the subsequent agent(s).

Pharmaceutical compositions and formulations for use in treatment of Type-I or Type-II diabetes include pharmaceutical compositions of a GPR75 agonist, alone or in combination with one or more additional therapeutic agents, in a mixture with a physiologically compatible carrier, which can be administered to a subject, for example, a human subject, for therapeutic or prophylactic treatment. Such GPR75 agonists include small molecules that function to induce the activity of GPR75.

In an embodiment, antibody molecules that bind to GPR75 may also be used to induce the activity of GPR75 in Type-I or Type-II diabetes subjects. “Antibody molecule” as used herein is intended to include intact antibodies, such as polyclonal antibodies or monoclonal antibodies (mAbs), as well as proteolytic fragments thereof such as the Fab or F(ab′)₂ fragments, chimeric antibodies, nanobodies, recombinant and engineered antibodies, single-chain antibodies and fragments thereof, as well as other molecules having at least one GPR75 antigen-binding site.

Also provided is a nanoparticle comprising a GPR75 agonist for use in treatment of Type-I or Type-II diabetes. Such nanoparticles can be natural or synthetic. They can be created from biological molecules or from non-biological molecules. In some cases, the GPR75 agonist is crosslinked to a polymer or lipid on nanoparticle surface. In embodiments, the GPR75 agonist is adsorbed onto the nanoparticle surface. In some embodiments, the GPR75 agonist is adsorbed onto the nanoparticle surface and then crosslinked to the nanoparticle surface. In some embodiments, the GPR75 agonist is encapsulated into the nanoparticle.

In particular embodiments, the nanoparticle is formed from a biocompatible polymer. Examples of biocompatible polymers include polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, or combinations thereof. In some cases, the nanoparticle is formed from a polyethylene glycol (PEG), poly(lactide-co-glycolide) (PLGA), polyglycolic acid, poly-beta-hydroxybutyrate, polyacrylic acid ester, or a combination thereof. In a specific embodiment the nanoparticle is a nanoliposome. Such nanoliposomes may be composed of phospholipids such as 1,2-distearoyl-sn-glycero-3-phosphocholine (DSPC), 1,2-dipalmitoyl-sn-glycero-3-phosphocholine (DPPC), 1,2-dimyristoyl-sn-glycero-3-phosphocholine (DMPC), 1,2-dioleoyl-sn-glycero-3-phosphocholine (DOPC), 1,2-distearoyl-sn-glycero-3-phospho-(1′-rac-glycerol) (DSPG), 1,2-dipalmitoyl-sn-glycero-3-phospho-(1′-rac-glycerol) (DPPG), 1,2-dimyristoyl-sn-glycero-3-phospho-(1′-rac-glycerol) (DMPG), 1,2-dioleoyl-sn-glycero-3-phospho-(1′-rac-glycerol) (DOPG), dipalmitoyl phosphatidylserine (DPPS), distearoyl phosphatidylserine (DSPS), dipalmitoyl phosphatidylinositol (DPPI), distearoyl phos phatidylinositol (DSPI), dipalmitoyl phosphatidic acid (DPPA), distearoyl phosphatidic acid (OSPA), 1,2-diacyl-3-trimethylammonium-propanes, (including but not limited to, dioleoyl (DOTAP), 1,2-dipalmitoyl-sn-glycero-3-phosphoethanolamine-N [methoxy(polyethylene glycol)-2000] (DPPE-PEG2000)), 1,2-distearoyl-sn-glycero-3-phosphoethanolamine-N-[methoxy(polyethylene glycol)-1000] (DSPE-PEG2000), and cholesterol.

In some embodiments, the GPR75 agonist is coated on the nanoparticle using a crosslinking agent. In some embodiments, the GPR75 agonist is adsorbed onto the nanoparticle surface. In some embodiments, the GPR75 agonist is adsorbed onto the nanoparticle surface followed by covalent crosslinking of the GPR75 agonist to the nanoparticle surface using a crosslinking agent.

Crosslinking agents suitable for crosslinking the GPR75 agonist to produce the nanoparticle, or to coat SC-membrane protein on the nanoparticle are known in the art, and include those selected from the group consisting of formaldehyde, formaldehyde derivatives, formalin, glutaraldehyde, glutaraldehyde derivatives, a protein cross-linker, a nucleic acid cross-linker, a protein and nucleic acid cross-linker, primary amine reactive crosslinkers, sulfhydryl reactive crosslinkers, sulfydryl addition or disulfide reduction, carbohydrate reactive crosslinkers, carboxyl reactive crosslinkers, photoreactive crosslinkers, cleavable crosslinkers, AEDP, APG, BASED, BM(PEO)3, BM(PEO)4, BMB, BMDB, BMH, BMOE, BS3, BSOCOES, DFDNB, DMA, DMP, DMS, DPDPB, DSG, DSP, DSS, DST, DTBP, DTME, DTSSP, EGS, HBVS, sulfo-BSOCOES, Sulfo-DST, and Sulfo-EGS.

Pharmaceutical compositions and formulations for use in treatment of Type-I or Type-II diabetes include pharmaceutical compositions comprising an effective amount of a GPR75 agonist and a physiologically compatible carrier, which can be administered to a subject, for example, a human subject, for therapeutic or prophylactic treatment of Type-I or Type-II diabetes. As used herein, “physiologically compatible carrier” refers to a physiologically acceptable diluent including, but not limited to water, phosphate buffered saline, or saline, and, in some embodiments, can include an adjuvant. Acceptable carriers, excipients, or stabilizers are nontoxic to recipients at the dosages and concentrations employed, and can include buffers such as phosphate, citrate, and other organic acids; antioxidants including ascorbic acid, BHA, and BHT; low molecular weight (less than about 10 residues) polypeptides; proteins, such as serum albumin, gelatin or immunoglobulins; hydrophilic polymers, such as polyvinylpyrrolidone, amino acids such as glycine, glutamine, asparagine, arginine, or lysine; monosaccharides, disaccharides, and other carbohydrates including glucose, mannose, or dextrans; chelating agents such as EDTA; sugar alcohols such as mannitol or sorbitol; salt-forming counter-ions such as sodium; and/or nonionic surfactants such as Tween, Pluronics, or PEG. Adjuvants suitable for use with the presently disclosed compositions include adjuvants known in the art including, but not limited to, incomplete Freund's adjuvant, aluminum phosphate, aluminum hydroxide, and alum.

The presently disclosed pharmaceutical compositions can be administered using a variety of methods known in the art. More particularly, as described herein, the GPR75 agonist can be administered to a subject for treatment of Type-I or Type-II diabetes by any suitable route of administration, including orally, nasally, transmucosally, parenterally, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections, intracisternally, topically, as by powders, ointments, including buccally and sublingually, transdermally, through an inhalation spray, or other modes of delivery known in the art.

In some embodiments, the presently disclosed pharmaceutical compositions can be administered by rechargeable or biodegradable devices. For example, a variety of slow-release polymeric devices have been developed and tested in vivo for the controlled delivery of drugs. Suitable examples of sustained release preparations include semipermeable polymer matrices in the form of shaped articles, e.g., films or microcapsules. Sustained release matrices include polyesters, hydrogels, polylactides (U.S. Pat. No. 3,773,919; EP 58,481), copolymers of L-glutamic acid and gamma ethyl-L-glutamate (Sidman et al., Biopolymers 22:547, 1983), poly (2-hydroxyethyl-methacrylate) (Langer et al., J. Biomed. Mater. Res. 15:167, 1981), ethylene vinyl acetate (Langer et al., Id), or poly-D-(−)-3-hydroxybutyric acid (EP 133,988A). Sustained release compositions also include a liposomally entrapped GPR75 agonist, which can be prepared by methods known per se (Epstein et al., Proc. Natl. Acad. Sci. U.S.A. 82:3688, 1985; Hwang et al., Proc. Natl. Acad. Sci. U.S.A. 77:4030, 1980; U.S. Pat. Nos. 4,485,045 and 4,544,545; and EP 102,324A). Ordinarily, the liposomes are of the small (about 200-800 Angstroms) unilamelar type in which the lipid content is greater than about 30 mol % cholesterol, the selected proportion being adjusted for the optimal therapy. Such materials can include an implant, for example, for sustained release of the GPR75 agonist.

The presently disclosed subject matter also includes the use of a GPR75 agonist, in the manufacture of a medicament for treatment of Type-I or Type-II diabetes. Regardless of the route of administration selected, the GPR75 agonist pharmaceutical compositions are formulated into pharmaceutically acceptable dosage forms such as described herein or by other conventional methods known to those of skill in the art.

Actual dosage levels of the GPR75 agonist can be varied to obtain an amount of the active ingredient that is effective to achieve the desired therapeutic response for a particular subject, composition, route of administration, and disease, disorder, or condition without being toxic. The selected dosage level will depend on a variety of factors including the route of administration, the time of administration, the rate of excretion, the duration of the treatment, other drugs used in combination with the GPR75 agonist, the age, sex, weight, condition, general health and prior medical history of the patient being treated, and like factors well known in the medical arts.

A physician having ordinary skill in the art can readily determine and prescribe the effective amount of a GPR75 agonist-containing pharmaceutical composition required for treatment of Type-I or Type-II diabetes. For example, the physician could start doses of the GPR75 agonist lower than that required to achieve the desired therapeutic effect and gradually increase the dosage until the desired effect is achieved. Accordingly, the dosage range for administration will be adjusted by the physician, as necessary. It will be appreciated that an amount of a GPR75 agonist required for achieving the desired biological response, e.g., treatment or prevention of Type-I or Type-II diabetes, may be different from the amount of a GPR75 agonist effective for another purpose.

In general, a suitable daily dose of a GPR75 agonist will be that amount of the drug that is the lowest dose effective to produce a therapeutic effect. Such an effective dose will generally depend upon the factors described above. Effective dosages may be determined based generally on the weight of the subject to be treated. If desired, the effective daily dose of the GPR75 agonist can be administered as one, two, three, four, five, six or more sub-doses administered separately at appropriate intervals throughout the day, optionally, in unit dosage forms.

The presently disclosed compositions of a GPR75 agonist can be assembled into kits or pharmaceutical systems for use in treating or preventing Type-I or Type-II diabetes. In some embodiments, the presently disclosed kits or pharmaceutical systems include a GPR75 agonist in unit dosage form. In further embodiments, the GPR75 agonist can be present together with a pharmaceutically acceptable solvent, carrier, excipient, or the like, as described herein.

In some embodiments, the presently disclosed kits include one or more containers, including, but not limited to a vial, tube, ampule, bottle, and the like, for containing the GPR75 agonist. The one or more containers also can be carried within a suitable carrier, such as a box, carton, tube, or the like. Such containers can be made of plastic, glass, laminated paper, metal foil, or other materials suitable for holding medicaments.

The presently disclosed kits or pharmaceutical systems also can include associated instructions for using the GPR75 agonist containing compositions for treating Type-I or Type-II diabetes. In some embodiments, the instructions include one or more of the following: a description of pharmaceutical composition containing a GPR75 agonist; a dosage schedule and administration for treating or preventing Type-I or Type-II diabetes; precautions; warnings; indications; counter-indications; overdosage information; adverse reactions; animal pharmacology; clinical studies; and references. The instructions can be printed directly on a container (when present), as a label applied to the container, as a separate sheet, pamphlet, card, or folder supplied in or with the container.

Although the present invention and its advantages have been described in detail, it should be understood that various changes, substitutions, and alterations can be made herein without departing from the spirit and scope of the invention as defined in the claims below.

The present invention will be further illustrated in the following Examples, which are given for illustration purposes only and are not intended to limit the invention in any way.

Having thus described in detail preferred embodiments of the present invention, it is to be understood that the invention defined above is not limited to particular details set forth in the above description as many apparent variations thereof are possible without departing from the spirit or scope of the present invention.

### EXAMPLES

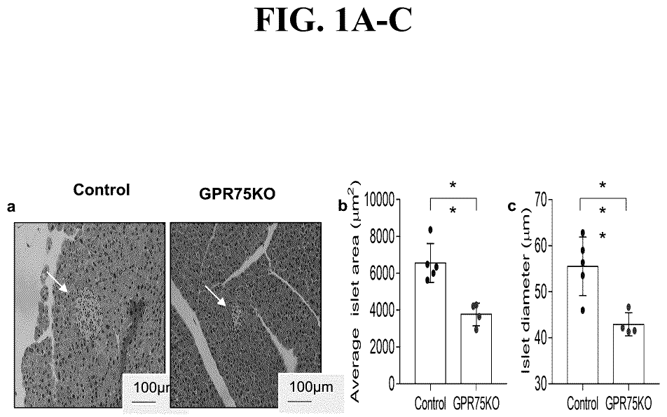

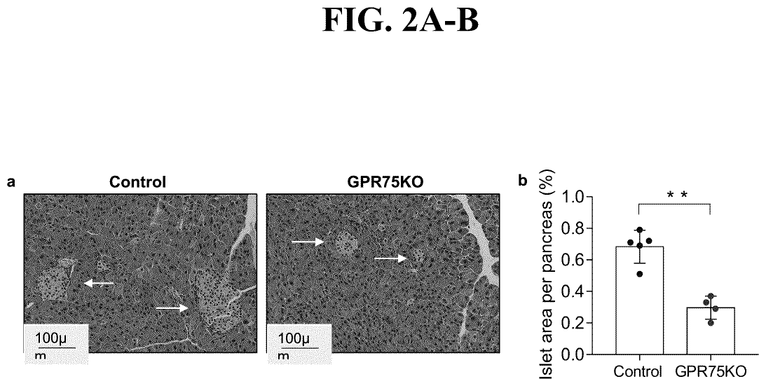

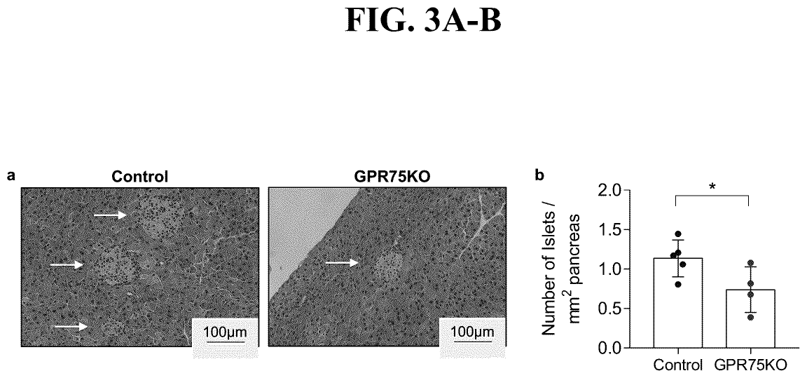

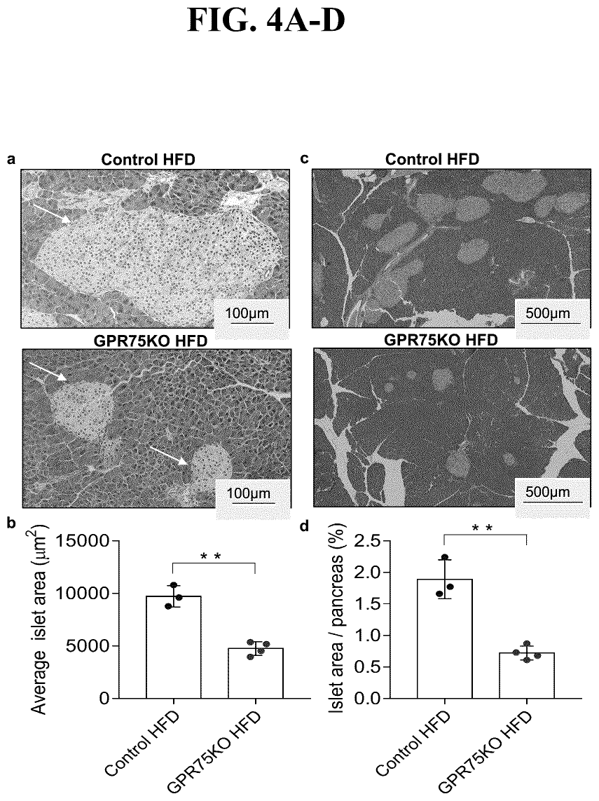

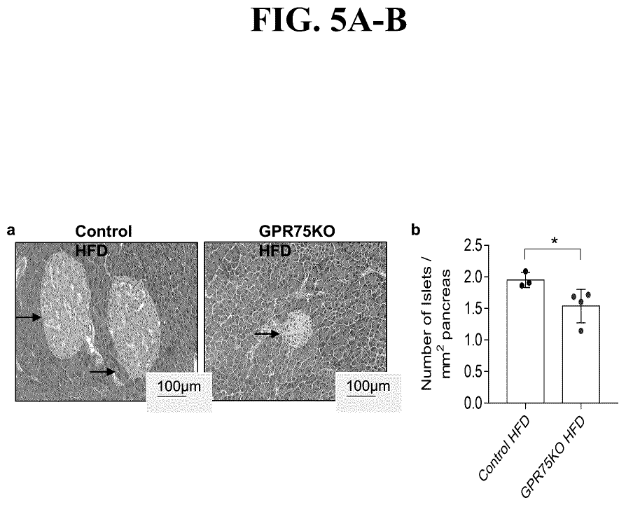

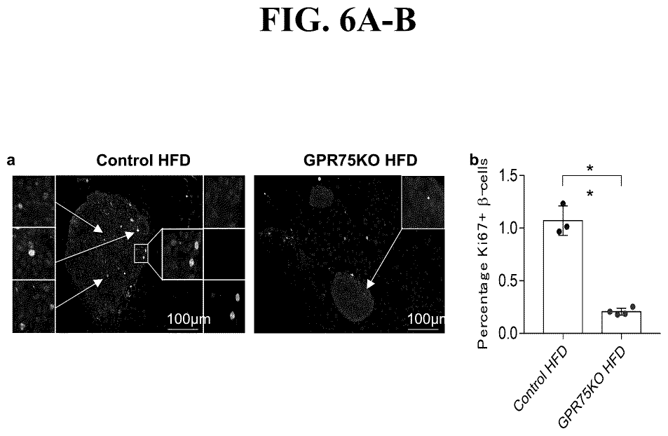

The effects of GPR75 knockout in ß-cells have been tested using a mouse model of whole-body GPR75 knockout. As demonstrated in FIGS. 1-6, analysis on harvested pancreases sections from these mice showed a significant decrease in islet size and number in mice without GPR75. Specifically, FIG. 1A-C shows that pancreatic islet size is reduced in GPR75KO mice. FIG. 2A-B shows that total pancreatic islet area is reduced in GPR75KO mice. FIG. 3A-B shows that total number of islets are reduced in GPR75KO mice. FIG. 4A-D shows that adaptive ß-cell mass is impaired in high-fat fed GPR75KO mice. FIG. 5A-B shows that the total number of islets are reduced in high-fat fed GPR75KO mice. In FIG. 6A-B immunofluorescence staining of pancreases obtained from high-fat fed mice with Ki67, a proliferation marker, showed a decrease in beta cell proliferation in the absence of GPR75.

What is claimed is:

1. A method of treating, or preventing diabetes in a subject in need thereof, the method comprising administration to the subject of a therapeutically effective dose of a GPR75 agonist.

2. The method of claim 1, wherein the diabetes is Type-I diabetes.

3. The method of claim 1, wherein the diabetes is Type-II diabetes.

4. The method of claim 1, wherein the GPR75 agonist is a small molecule.

5. The method of claim 1, wherein the GPR75 agonist is C-C Motif Chemokine Ligand 5 (CCL5) or 20-hydroxyeicosatetraenoic acid (20-HETE).

6. The method of claim 1, wherein the GPR75 agonist is an antibody.

7. The method of claim 1, further comprising administration of a second diabetes therapeutic agent.

8. A kit comprising: (i) one or more containers for containing a GPR75 agonist containing compositions; and (ii) instructions for using the GPR75 agonist containing compositions for treatment of diabetes.

9. The kit of claim 8, wherein the instructions include one or more of the following: (i) a description of pharmaceutical composition containing a GPR75 agonist; (ii) a dosage schedule; or (iii) instructions for administration of the pharmaceutical composition containing a GPR75 agonist for treatment of diabetes.
